(19) United States
(12) Patent Application Publication
(10) Pub. No.: US 2008/0280941 A1
(43) Pub. Date: Nov. 13, 2008

(54) 8-Phenoxy-Gamma Carboline Derivatives

(76) Inventor: Pierre Lourtie, Loos (FR)

Correspondence Address: KNOBBE MARTENS OLSON & BEAR LLP, 2040 MAIN STREET, FOURTEENTH FLOOR, IRVINE, CA, 92614, US

(21) Appl. No.: 11/579,162

(22) PCT Filed: Feb. 24, 2005
(86) PCT No.: PCT/EP05/01958

§ 371 Date: Dec. 5, 2006

(30) Foreign Application Priority Data
Mar. 5, 2004 (EP) 04 290 621.4
Jun. 28, 2004 (EP) 04 291 621.3

(51) Int. Cl. A61K 31/437; C07D 471/04; A61P 11/06; A61P 11/00; A61P 25/06; A61P 19/02; A61P 1/00; A61P 17/06

(52) U.S. Cl. 514/292; 546/86

(57) ABSTRACT

Compounds of Formula I and corresponding pharmaceutical compositions are disclosed.

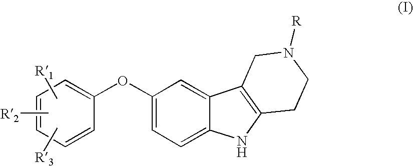

### FIELD OF THE PRESENT INVENTION

The present invent relates to novel 8-phenoxy-γ-carboline derivatives of the Formula (I), pharmaceutical compositions containing same and their application in the treatment of indications relating to neurogenic inflammation:

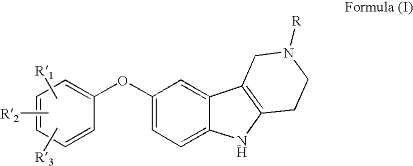

wherein R, R′₁, R′₂, R′₃ are as defined in claim 1.

### DESCRIPTION OF THE PRIOR ART

Inflammation, due to the stimulation of sensory nerves (fibers C) can be inhibited by agonists of the receptor 5HT1, for example specific indole derivatives. According to the literature, this reaction is mediated by the activation of pre-synaptic receptors of the type 5HT1D, 5HT1B and/or 5HT1F, and the subsequent inhibition of liberation of neuropeptides stimulating inflammation (substance P and CGRP) in the peripheral region.

The French patent application FR 2814166 describes specific derivatives of 5-phenoxyindole, capable of the inhibition of neurogenic inflammation by a mechanism independent from the activation of receptors 5HT1B and 5HT1D.

EP-A-0 905 136 discloses derivatives of carboline which show an affinity for serotonine receptors. These compounds however do not possess a phenoxy group at the position 8 of the carboline nucleus. Furthermore the document discloses that the compounds described therein do act by means of interaction with receptors 5HT₁ or 5HT₂.

Several further derivatives of γ-carboline having therapeutical applications have been described up-to-date, in particular in relation of their tranquilizing, psychotropic or anti-psychotic properties. Furthermore, derivatives of γ-carboline have also been proposed as agonists or antagonists for serotoninergic receptors, like the receptors of the type 5HT2 (WO 00/770001, WO 00/770002, WO 00/770010 and WO 99/12926).

However, those compounds do not possess a phenoxy group at the position 3 of the γ-carboline structure. Furthermore these documents clearly reveal that the pharmacological activity is associated with the interaction of receptors of the type 5HT2.

WO 00/59904 discloses indole derivatives which are inhibitors of kinase p38α. These compounds however do not comprise a carboline nucleus comprising a phenoxy group at the position 8 thereof.

WO 00/12074 discloses, like the aforementioned document, inhibitors of kinase p38α. These compounds however again do not comprise a carboline nucleus comprising a phenoxy group at the position 8 thereof.

U.S. Pat. No. 6,177,440 discloses tricyclic compounds which are employed as inhibitors with respect to the liberation of fatty acids. This application in particular is useful for the treatment of septic shocks.

All the prior art discussed above, however, fails to disclose any information which could be regarded as suggesting anything with respect to compounds having the capability to treat neurogenic inflammation by means of a mechanism independent from the activation of the above mentioned receptors.

### OBJECT OF THE PRESENT INVENTION

However, there remains still a desire for compounds capable of treating neurogenic inflammation by means of a mechanism independent from the activation of the above mentioned receptors.

It now has been discovered in the context of the present invention that also specific derivatives of γ-carboline possess the unique property of inhibiting neurogenic inflammation also by a mechanism independent from the activation of receptors 5HT1B and 5HT1D.

Contrary to the perception in the art, the present inventors have discovered that 8-phenoxy-γ-carboline derivatives are also inhibitors of neurogenic inflammation by a mechanism independent from the activation of the receptors 5HT1B and 5HT1D. The present invention has been made based on the above findings.

### BRIEF DESCRIPTION OF THE PRESENT INVENTION

The present invention provides compounds of the Formula (I)

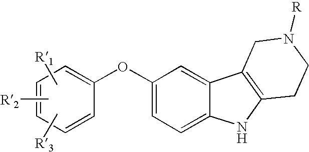

wherein
R represents hydrogen, a C₁-C₆ alkyl group, a C₁-C₆ haloalkyl group, a C₆-C₁₈ aryl group, a C₆-C₁₈ aryl-C₁-C₆ alkyl group, a heterocyclic group, a heterocycle-C₁-C₆ alkyl group, or a group wherein R forms with any one of the two carbon atoms adjacent to the nitrogen atom to which R is bound a condensed cyclic group, and
wherein the alkyl group, the aryl group, the aralkyl group, the heterocyclic group and the condensed cyclic group may be substituted by one or more groups, chosen independently from halogens, hydroxy, amino, monoalkylamino, dialkylamino, amido, N-alkyl amido, N,N-dialkyl amido, nitro, cyano, —COOH, —COO(C₁-C₄ alkyl), —OCF₃, —SO₂(C₁-C₄ alkyl), C₁-C₆ alkyl, C₁-C₆ alkoxy, phenoxy, C₁-C₆ alkoxy carbonyl, C₁-C₆ acyloxy, C₁-C₆ hydroxyalkyl, C₁-C₆ alkoxy-C₁-C₆ alkyl, C₁-C₆ alkyl carbonyl, and a residue comprising a linear chain with from 3 to 13 atoms selected from C and O, terminated with an aryl group.
R′₁, R′₂ and R′₃ are independently chosen from hydrogen, halogens, hydroxy, nitro, cyano, —COOH, —COO(C₁-C₄ alkyl), —OCF₃, —SO₂(C₁-C₄ alkyl), C₁-C₆ alkyl, C₁-C₆ alkoxy, phenoxy, C₁-C₆ alkoxy carbonyl, C₁-C₆ acyloxy, C₁-C₆ hydroxyalkyl, C₁-C₆ alkoxy-C₁-C₆ alkyl and C₁-C₆ alkyl carbonyl,
and pharmaceutically acceptable salts thereof.

If any of the primary groups defined for the residues R, R′₁, R′₂ and R′₃ comprise substituents it is preferred that they comprise from one to three substituents as defined above, preferably one or two and most preferably one substituent. Preferred substituents are hydrogen, halogens, hydroxy, amino, monoalkylamino, dialkylamino, amido, N-alkyl amido, —COOH, —COO(C₁-C₄ alkyl), and a residue comprising a linear chain with from 3 to 13 atoms selected from C and O, terminated with an aryl group.

As used throughout the present application, the term “alkyl” describes a straight chain or branched hydrocarbon radical comprising preferably from 1 to 6 carbon atoms, more preferably from 1 to 4 carbon atoms. Examples of alkyl groups are methyl, ethyl, propyl, isopropyl, butyl, tert-butyl, isobutyl, pentyl and hexyl.

The term “alkyl” may also designate a cycloalkyl group, i.e. a cyclic hydrocarbon radical having preferably up to 6 carbon atoms, as for example cyclopropyl, cyclobutyl, cyclopentyl or cyclohexyl.

A radical of the type heterocyclic group defines a carbocycle wherein at least one of the carbon atoms has been replaced by at least one hetero atom selected among O, N or S. The carbocycle may be saturated or unsaturated. The above definition applies also with respect to the heterocycle of the heterocycle-alkyl group. One example of a heterocycle is in particular the radical piperidinyl.

The term “halogen” defines in particular a bromine atom, a chlorine atom, an iodine atom or a fluorine atom, wherein fluorine, chlorine and bromine are preferred.

The term “haloalkyl” defines an alkyl radical as defined above substituted by at least one halogen atom, preferably a fluorine atom or a chlorine atom, or preferably a bromine atom. The halo alkyl groups also comprise perfluorated alkyls, i.e. groups of the general formula CₙF₂ₙ₊₁, wherein n represents 1 to 6, preferably 1 to 5.

The term “aryl” defines a hydrocarbon group which is aromatic and which may be monocyclic or polycyclic and which comprises preferably from 6 to 18 carbon atoms in the ring, or preferably from 6 to 10 carbon atoms. As examples of the aryl group, phenyl, naphtyl, tetrahydronaphtyl, indanyl, biphenyl, can be named. Phenyl and naphtyl are preferred. The term aryl group also comprises aryl groups wherein one or more of the ring carbon atoms have been replaced with one or more hetero atoms, including S, O and N. These rings are also termed heteroaromatic groups, such as pyridinyl.

The term “aryl alkyl” or “aralkyl” defines an alkyl chain substituted with an aryl group, wherein the alkyl groups and the aryl groups are defined as above. Preferably the aryl group is located at the terminal carbon atom of the alkyl group. One example of such a group is the residue benzyl. This principle applies also to the heterocycle-alkyl groups, i.e. these residues comprise a heterocycle bound to the molecule by means of an alkyl group.

The term “alkoxy” defines an alkyl group as defined above, bound to an oxygen atom, wherein the oxygen atom provides the link to the molecule substituted with the alkoxy group. Examples thereof are the radicals methoxy, ethoxy, propyloxy, isopropyloxy, butoxy and hexyloxy.

The term “hydroxyalkyl” defines an alkyl group as defined above, comprising at least one hydroxy group, preferably 1 to 4 hydroxy groups, most preferably one hydroxy group. Preferably the hydroxy group is present at the terminal carbon atom of the alkyl group.

The term “alkoxy carbonyl” defines a moiety —C(═O)—O-alkyl, wherein alkyl is as defined above. The term “acyloxy” defines a moiety —O—C(═O)-alkyl, wherein alkyl is as defined above. The term “alkoxy alkyl” defines a moiety -alkyl-O-alkyl, wherein alkyl is as defined above. The term “alkyl carbonyl” defines a moiety —C(═O)-alkyl, wherein alkyl is as defined above.

The condensed cyclic group, formed by R and the nitrogen atom to which R is bound and the adjacent carbon atom is preferably a six- or five-membered cyclic structure, including the nitrogen atom to which R is bound and the adjacent carbon atom. Preferably this cyclic group comprises no further hetero atom in addition to the nitrogen atom to which R is bound.

Preferred are compounds wherein R is selected among hydrogen, alkyl, aralkyl, heterocyclic and heterocycle alkyl, optionally substituted as defined above. Further preferred for R are hydrogen, alkyl, and aralkyl, preferably substituted with one substituent selected among hydroxy, —COOH, —COO(C₁-C₄alkyl), N-alkyl amido, amino, monoalkylamino, or dialkylamino. More preferred are compounds wherein R is selected among hydrogen, benzyl and alkyl, preferably alkyl substituted at the terminal carbon atom with one substituent selected among hydroxy, —COOH, —COO(C₁-C₄ alkyl), N-alkylamido, amino, monoalkylamino, or dialkylamino.

Further preferred embodiments of compounds of the Formula (I) are compounds wherein the group R represents a benzyl group, optionally substituted with one or more substituents, selected from the group comprising halogen, nitro, cyano, —COOH, —COO(C₁-C₄ alkyl), —OCF₃, SO₂(C₁-C₄ alkyl), C₁-C₆ alkyl, C₁-C₆ alkoxy, C₁-C₆ alkoxy carbonyl, C₁-C₆ acyloxy, C₁-C₆ hydroxyalkyl, C₁-C₆ alkoxy-C₁-C₆ alkyl, and C₁-C₆ alkyl carbonyl, a residue comprising a linear chain with from 3 to 13 atoms selected from C and O, terminated with an aryl group. In particular, preferred are compounds wherein the phenyl group of the benzyl moiety is substituted with one or more groups chosen from the group comprising alkyl, perfluoroalkyl, in particular trifluoromethyl, alkoxy, for example methoxy or phenoxy, halogen, preferably chlorine and fluorine, —COOH, —COO(C₁-C₄ alkyl), nitro and cyano.

Further preferred embodiments of this compounds are compounds wherein the aryl group of the residue comprising a linear chain with from 3 to 13 atoms selected from C and O, terminated with an aryl group is substituted with one or more groups chosen from the group comprising alkyl, perfluoroalkyl, in particular trifluoromethyl, alkoxy, for example methoxy or phenoxy, halogen, preferably chlorine and fluorine, —COOH, —COO(C₁-C₄ alkyl), nitro and cyano. Preferred are two substituents, in particular two trifluoromethyl groups. Preferably the aryl moiety of this residue is a phenyl group, preferably with two substituents as defined above, preferably located at the two meta-positions.

If the residue R represents a benzyl group, preferably a non-substituted benzyl group, the groups R′₁, R′₂ and R′₃ preferably independent from one and each other represent a hydrogen atom, a halogen atom or an alkyl group, wherein hydrogen is in particular preferred.

The residue comprising a linear chain with from 3 to 13 atoms selected from C and O terminated with an aryl group, preferably is selected from the group of residues wherein the linear chain comprises 1 oxygen atom and 2 alkylene groups each having independently from 1 to 6 carbon atoms. The two alkylene groups preferably have each independently from 1 to 4 carbon atoms and more preferably 1 or 2 and most preferably 1 carbon atom. The most preferred embodiment is a linear chain comprising 3 atoms, 2 carbon atoms and 1 oxygen atom which lies between the 2 carbon atoms. In connection with the residue comprising a linear chain with from 3 to 13 atoms selected from C and O, it is more preferred if the alkylene groups are not substituted, i.e. the carbon atoms are each only connected to 2 hydrogen atoms so that saturated, unsubstituted residues arise.

The preferred linear chain portion of this residue in this connection can also be depicted as follows:

—(CH₂)ₙ—O—(CH₂)ₖ—

wherein n and k each are independently selected from a number of from 1 to 6 and wherein n and k preferably are identical and wherein n and k preferably are each 1 or 2 and most preferably 1.

The aryl group terminating linear chain of the residue in accordance with this preferred embodiment may be selected from the aryl groups as defined above. The most preferred aryl group in this respect is a phenyl group. The aryl group and in particular the phenyl group may be substituted as outlined above in connection with the residue comprising a linear chain with from 3 to 13 atoms selected from C and O, terminated with an aryl group. The most preferred embodiment in this connection is a phenyl group substituted with two trifluoromethyl groups at the meta-positions of the phenyl ring.

The residue comprising a linear chain with from 3 to 13 atoms selected from C and O, terminated with an aryl group (and also the therewith associated preferred embodiments) are preferred in particular if the group R represents a benzyl group. In such an embodiment, the residue comprising a linear chain from 3 to 13 atoms selected from C and O, terminated with an aryl group replaces one of the hydrogen atoms of the CH₂ group of the benzyl residue. One example of such a preferred compound is depicted below.

In this embodiment, it is furthermore preferred if the benzyl group does not comprise any further substituents, while the groups R′₁, R′₂ and R′₃ may be selected from the groups as defined below. However, it is also preferred in this embodiment if the groups R′₁, R′₂ and R′₃ are each hydrogen.

The compounds in accordance with the above-described preferred embodiment, i.e. wherein the group R represents a benzyl group substituted with one residue as defined above, are particularly potent compounds with respect to the treatment of inflammations of the respiratory system, in particular asthma. Without being wanted to be restricted to a particular theory, it is assumed that the specific architecture of the compounds in accordance with this preferred embodiment, comprising a group R with two aromatic residues, enables a strong interaction with receptors important for the indication mentioned above. It is in particular assumed that the specific construction of the group R in accordance with this preferred embodiment enables a strong interaction with the receptors of the type NK, in particular NK1, so that a potent activity is ensured. Since these compounds also comprise the 8-phenoxy residue, it is assumed that these compounds also act against the receptors generally associated with neurogenic inflammation. Accordingly, these preferred compounds in accordance with the present invention represent hybrid molecules enabling an activity with respect to two different types of receptors, which shows that the compounds of this preferred embodiment in accordance with the present invention must be regarded as highly valuable compounds having a high potential in particular in the treatment of inflammations of the respiratory system, in particular asthma.

Preferred examples for the groups R′₁, R′₂ and R′₃ are independently selected from hydrogen, halogens, alkyl, nitro, cyano, —COOH and alkoxy, more preferably hydrogen halogens and alkyl. It is preferred that either R′₁, R′₂ and R′₃ are all hydrogen or that two of them are hydrogen while the third one is selected among any of the substituents defined above for R′₁, R′₂ and R′₃ with the exception of hydrogen. The preferred embodiments as defined above for R′₁, R′₂ and R′₃ also are valid with respect to the case that only one of R′₁, R′₂ and R′₃ is not hydrogen.

The salts of the compounds in accordance with the present invention with acids or bases are also comprised within the present invention. The acids and bases may be inorganic acids or bases or organic acids and bases and the only requirement in this respect is that the acids and bases are pharmaceutically acceptable. Examples of salts with pharmaceutically acceptable acids are hydrochlorides, hydrobromides, sulfates, acetates, hydrogenosulfates, dihydrogenophosphates, methanesulfonates, methylsulfates, maleates, fumarates, sulfonates, 2-naphtalenesulfonates, glycolates, gluconates, citrates, benzoates, salicylates, ascorbates, tartrates, succinates, lactates, glutarates, toluenesulfonates, ascorbates and oxalates. As examples of salts with inorganic or organic bases, salts of the ammonium type can be cited and salts with alkaline metals, such as sodium or potassium or lithium, or salts with alkaline earth metals, such as calcium, magnesium or other suitable metals.

Preferred compounds in accordance with the present invention are as follows:
phenyl-2,3,4,5,-tetrahydro-1H-pyridol[4,3-b]indol-8-yl ether; (8-phenoxy-2,3,4,5-tetrahydro-1H-pyrido[4,3-b]indole),
N-benzyl-phenyl-2,3,4,5,-tetrahydro-1H-pyrido[4,3-b]indol-8-yl ether; (8-phenoxy-2-N-benzyl-2,3,4,5-tetrahydro-1H-pyrido[4,3-b]indole),
8-phenoxy-N-benzyl-2,3,4,5-tetrahydro-5H-pyridinium[4,3-b]indole-hydrochloride.

Other preferred corn pounds are compounds selected among the following structures:

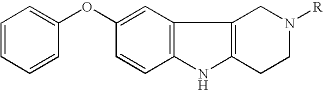

| | | | |
|---|---|---|---|
| R = | —H | R = | 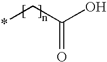 |
| R = | 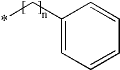 | R = | 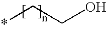 |
| R = | 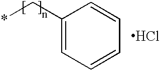 | R = | 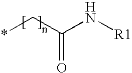 |
| R = | 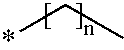 | R = | 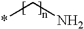 |
| R = | 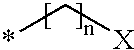 | R = | 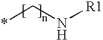 |
| R = | 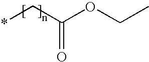 | R = | 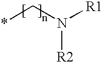 |

WHEREIN N IS FROM 1 TO 6, WHEREIN R1 AND R2 REPRESENT ALKYL AND WHEREIN X REPRESENTS HALOGEN

A further preferred example of a compound in accordance with the present invention is as follows:

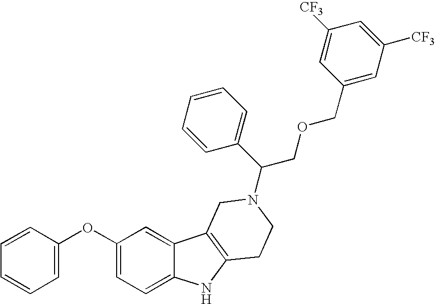

In particular preferred are compounds according to the following

R′₁, R′₂ and R′₃ are hydrogen or R′₁, R′₂ are hydrogen and R′₃ is halogen, alkyl, nitro, cyano, —COOH or alkoxy and R is alkyl, optionally substituted at the terminal carbon atom with hydroxy, —COOH, —COO(C₁-C₄ alkyl), N-alkyl amido, amino, monoalkylamino, dialkylamino.

R′₁, R′₂ and R′₃ are hydrogen or R′₁, R′₂ are hydrogen and R′₃ is halogen, alkyl, nitro, cyano, —COOH or alkoxy and R is aralkyl, optionally substituted with hydroxy, —COOH, —COO(C₁-C₄ alkyl), N-alkyl amido, amino, monoalkylamino, dialkylamino.

R′₁, R′₂ and R′₃ are hydrogen or R′₁, R′₂ are hydrogen and R′₃ is halogen, alkyl, nitro, cyano, —COOH or alkoxy and R is hydrogen.

R′₁, R′₂ and R′₃ are all not hydrogen and R is alkyl, optionally substituted at the terminal carbon atom with hydroxy, —COOH, —COO(C₁-C₄ alkyl), N-alkyl amido, amino, monoalkylamino, dialkylamino.

R′₁, R′₂ and R′₃ are all not hydrogen and R is aralkyl, optionally substituted with hydroxy, —COOH, —COO(C₁-C₄alkyl), N-alkyl amido, amino, monoalkylamino, dialkylamino.

### Methods of Preparation for Compounds of Formula (I)

Generally the compounds of Formula (I) may be prepared based on chemical reaction known to the average skilled person for chemical synthesis of carboline compounds, in particular methods based on Fischer-Synthesis.

### Therapeutical Application

The compounds of the present invention are promising active principles for the treatment and/or prophylaxis of diseases associated with neurogenic inflammation, in particular implications with respect to sensory nerves of the type fiber C. In this connection chronic as well as acute inflammations may be mentioned, such as rheumatic polyarthritis, asthma, skin irritations, such as psoriasis, urtikaria, vascular disorders, such as venous insufficiency, hemorrhoidal disorders, urologic disorders, such as cystitis and incontinence, as well as migraine and pain.

The present invention provides pharmaceutical compositions comprising an active compound of formula (I) or a pharmaceutically acceptable salt thereof together with a pharmaceutically acceptable carrier, and, optionally, further additives known in the art. The formulation of such a composition (amount of active compound, type of carrier and further additives) depends upon the active compound selected, the patient to be treated (age, weight and constitution), the mode of administration, the medicinal indication and other factors known to the practioner. The pharmaceutical compositions of the present invention comprise compositions for parenteral, oral, rectal, percutaneous as well as permucosal administration.

Pharmaceutical compositions of the present invention may be prepared in the form of solutions or suspensions for injection in multi dosis vials, in the form of tablets which may be coated further, in the form of dragees, capsules, capsules of gelatine, pills, powders, suppositories or rectal capsules, solutions or suspensions, emulsions, gels and cremes, as well as in the form of an aerosol or a pomade.

The term pharmaceutically acceptable carrier mentioned above all excipients which do not give rise to undesired or allergic reactions when compounded with the active principle and when administered to the patient in need, human or animal.

As example of typical excipients cellulose derivatives as well as microcrystalline cellulose, alkaline earth carbonates, magnesium or potassium phosphate, starches, modified starches, lactose, glucose and others may be cited, in particular for solid formulations.

For rectal uses suitable carriers (excipients) are cacao butter or polyethylene glycol stearates.

For parenteral use, water, aqueous solutions, physiological serum, isotonic solutions are suitable and preferred excipients.

Based on his common knowledge the average skilled person, however, is in the position to determine suitable excipients which are capable of fulfilling the desired function of a pharmaceutically acceptable carrier in a given composition of the present invention.

The present invention furthermore provides the use of a compound of formula (I) or a pharmaceutically acceptable salt thereof for the preparation of a medicament for the treatment and/or prevention of a neurogenous inflammation, in particular venous insufficiency, hemorrhoidal inflammations, urologic disorders, pain, migraine and skin irritations.

The invention also provides methods for treating and/or preventing neurogenous inflammations, in particular those cited above, in a patient, wherein the method comprises the administration of an effective amount of a compound of formula (I) or a pharmaceutically acceptable salt thereof to a patient in need of such a treatment.

The term “effective amount” intends to designate an amount sufficient to allow prevention and/or treatment of a disorder associated with neurogenous inflammation.

The invention will be illustrated further by means of the following examples which are not to be construed as restricting the invention. In these examples:
Melting points: determined with a capillary type apparatus of METTLER.
Thin layer chromatography (Rf): obtained with plates of silica gel containing an UV fluorescence indicator UV₂₅₄ at a thickness of 0.25 mm. Solvents used are indicated for each compound.
Mass spectra (SM): obtained with a spectrometer of the type AEI MS-50 or with a spectrometer of the type FISONS VG PLATFORM. The mode of ionisation is indicated for each analysis.
NMR spectra: ¹H and du ¹³C NMR were realized with a BRUCKER model at 250 MHz and 62.5 MHz, respectively. The deuterated solvents used are indicated for each analysis.
IR spectra: obtained with a NICOLET Impact 410 at a concentration of 1% (m/m) dispersed in KBr.
Microanalysis: microanalysis of C,H,N were obtained by means of measurements of thermal conductivity in a manner known to the skilled person. O and S were determined by coulometry and Cl was determined by potentiometry.

### EXAMPLE 1

### 8-Phenoxy-2,3,4,5-tetrahydro-1H-pyrido[4,3-b]indole

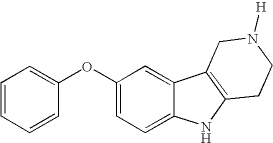

To a suspension of 28.00 g (0.121 mol) 4-phenoxyhydrazine in 834 ml ethanol/HCl (4 mol/L) at 20° C., 22.60 g (0.144 mol) of the hydrochloride of 4-piperidone were added. The reaction medium turned beige/rose and was heated at reflux (84° C.) for 6 hours. After cooling to room temperature the orange suspension was filtered. The precipitate was recrystallized in methanol and washed with dichloromethane. The white powder which was obtained was subjected to ultrasonic treatment and dried for 50 hours in vacuum. Subsequently the powder was dissolved in water and filtered with a millipore filter. The filtrate was rendered basic using an aqueous solution of ammonia (32%), until pH 8, and the suspension thus obtained was filtered. The white precipitant was dried in vacuum for 16 hours and 13.35 g of 8-phenoxy-2,3,4,5-tetrahydro-1H-pyrido[4,3-b]indole in the form of a white powder were obtained.

Yield: 41%

Mp: 179° C.

Rf: 0.22 (CH₂Cl₂/MeOH, 98/2)

SM: (APCI⁺): m/z 265 (M+H)⁺, 261 (M-NH—OC₆H₅+H)⁺

(APCI⁻): m/z 263 (M−H)⁻, 259 (M-NH—OC₆H₅−H)⁻

¹H-NMR (DMSO D6): δ (ppm)

10.78 (s, 1H, H-5)

7.25-7.32 (m, 3H, H-6, H-3′, H-5′)

6.95-7.01 (m, 2H, H-9, H-4′)

6.85 (d, 2H, H-2′, H-6′, J_{H2′-H3}=J_{H6′-H5′}=7.8 Hz)

6.71 (dd, 1H, H-7, J_{H7-H6}=8.6 Hz, J_{H7-H9}=2.3 Hz)

3.76 (s, 2H, H-1)

2.98 (pt, 2H, H-4)

2.65 (pt, 2H, H-3)

¹³C-NMR (DMSO D6): δ (ppm)

159.9 (1C, C-1′)

148.7 (1C, C-8)

135.7 (1C, C-4-a)

133.1 (1C, C-5a)

130.2 (2C, C-3′, C-5′)

126.9 (1C, C-9a)

122.3 (1C, C-4′)

117.2 (2C, C-6′, C-2′)

114.0 (1C, C-7)

112.2 (1C, C-6)

109.1 (1C, C-9b)

108.7 (1C, C-9)

43.5 (1C, C-3)

42.2 (1C, C-1)

24.8 (1C, C-4)

IR (KBr): ν (cm⁻¹)

3413 (NH piperidine), 3034 (NH indol), 2907 and 2855 (CH₂), 1586 (NH), 1489 (CH₂),1237 and 1222 (C—O—C and C—C—N)

Microanalysis: C₁₇H₁₆N₂O

Theory % C, 77.25; % H, 6.10; % N, 10.60; % O, 6.05

Measured % C, 77.10; % H, 5.99; % N, 10.65; % O, 6.21

### EXAMPLE 2

### 8-Phenoxy-N-benzyl-2,3,4,5-tetrahydro-1H-pyrido[4,3-b]indole

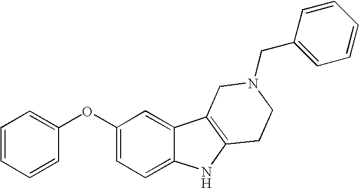

To a suspension of 1.00 g (3.79 mmol) 8-phenoxy-2,3,4,5-tetrahydro-1H-pyridido[4,3-b]indole in 180 ml anhydrous tetrahydrofurane under a nitrogen atmosphere at 26° C. were added 1.42 g (6.70 mmol) sodium triacetoxyborohydride and 0.40 ml (3.93 mmol) of benzaldehyde. The white reaction mixture was stirred for 18 hours and 50 minutes. The yellow suspension obtained was washed with a saturated solution of sodium hydrogenocarbonate. The organic phase was dried over sodium sulfate, filtered and concentrated under reduced pressure in order to obtain a yellow foamy mass. This mass was purified by means of flash chromatography (support: silica 60 μm, h=16 cm, d=4 cm; eluant: dichloromethane/methanol/aqu.ammonia 98/1.9/0.1). The yellow foamy mass obtained was treated with ultrasonic and dried in vacuum for six days in order to obtain 0.01 g of 8-phenoxy-N-benzyl-2,3,4,5-tetrahydro-1H-pyrido[4,3-b]indole in the form of a pale yellow powder.

Yield: 75%

Mp: 102.3° C.

Rf: 0.26 (CH₂Cl₂/MeOH/NH_{3aq}, 97/2.9/0.1)

SM: (APCI⁺): m/z 355 (M+H)⁺, 261 (M-OC₆H₅+H)⁺

: (APCI⁻): m/z 353 (M−H)⁻, 259 (M-OC₆H₅−H)⁻

¹H-NMR (DMSO D6): δ (ppm)

10.84 (s, 1H, H-5)

7.21-7.37 (m, 8H, H-6, H-3′, H-5′, H-3″, H-5″, H-4″, H-2″, H-6″)

6.98 (t, 1H, H-4′, J_{H4′-H3′}=J_{H4′-H5′}=7.3 Hz)

6.91 (d, 1H, H-9, J_{H9-H7}=2.1 Hz)

6.85 (d, 2H, H-2′, H-6′, J_{H2′-H3′}=J_{H6′-H5′}=7.8 Hz)

6.72 (dd, 1H, H-7, J_{H7-H6}=8.6 Hz, J_{H7-H9}=2.3 Hz)

3.70 (s, 2H, H-a)

3.52 (s, 2H, H-1)

2.77 (s, 4H, H-3, H-4)

¹³C-NMR (DMSO D6): δ (ppm)

159.3 (1C, C-1′)

148.4 (1C, C-8)

138.9 (1C, C-1″)

134.6 (1C, C-4-a)

133.1 (1C, C-5a)

129.8 (2C, C-3′, C-5′)

128.8 (2C, C-3″, C-5″)

128.3 (2C, C-2″, C-6″)

127.0 (1C, C-4″)

126.3 (1C, C-9a)

121.9 (1C, C-4′)

116.8 (2C, C-6′, C-2′)

113.6 (1C, C-7)

111.9 (1C, C-6)

107.9 (1C, C-9b)

107.5 (1C, C-9)

61.6 (1C, C-a)

49.9 (1C, C-3)

49.4 (1C, C-1)

23.7 (1C, C-4)

IR (KBr): ν (cm⁻¹)

3409 and 3034 (NH), 3062 and 3035 (CH aromatic), 2807 (CH₂), 1593 (C═C), 1473 (CH₂), 1240 and 1224 (C—O—C and C—C—N)

Microanalysis: C₂₄H₂₂N₂O

Theory % C, 81.33; % H, 6.26; % N, 7.90; % O, 4.51

Measured % C, 81.50; % H, 6.28; % N, 7.86; % O, 4.65

### EXAMPLE 3

### 8-Phenoxy-N-benzyl-2,3,4,5-tetrahydro-5H-pyridinium[4,3-b]indole-hydrochloride

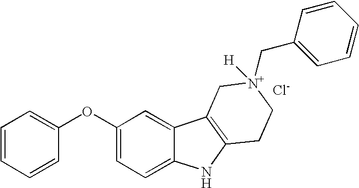

To a suspension of 8.00 g (34.0 mmol) 4-phenoxy-phenyl hydrazine in 250 ml hydrochloric acid (4M), were added 7.41 ml (40.0 mmol) N-benzyl-4-piperidone. The orange reaction mixture was heated for 7 hours at reflux. The reaction medium was removed in vacuum. The brown oil obtained was treated with a solution of sodium hydroxide in order to give a pH of 7. The solution was then extracted with dichloromethane. The organic phase was dried over sodium sulfate, filtered and concentated under reduced pressure. The yellow foamy mass obtained was purified by flash chromatography (support: silica 60 μm, h=16 cm; eluant: dichloromethane/methanol 97/3). The pale yellow foamy mass obtained was salted with of a solution of hydrochloric acid in diethyl ether. The formed precipitate was washed with ether and a cetonitrile. The white powder was dried in vacuum for six days in order to yield 3.71 g 8-phenoxy-N-benzyl-2,3,4,5-tetrahydro-1H-pyridinium[4,3b]indole-hydrochloride in the form of a white powder.

Yield: 69%

Mp: 226° C.

Rf: 0.26 (CH₂Cl₂/MeOH/NH₃aq, 97/2.9/0.1)

SM: (APCI⁺): m/z 355 (M+H)⁺, 261 (M-OC₆H₅+H)⁺

: (APCI⁻): m/z 353 (M−H)⁻, 259 (M-OC₆H₅—H)⁻

¹H-NMR (DMSO d6): δ (ppm)

11.32 (sl, 1H, NH⁺)

10.85 (s, 1H, H-5)

7.63 (m, 2H, H2″, H6″)

7.48 (m, 3H, H-3″, H-4″, H-5″)

7.38 (d, 1H, H-6, J_{H6-H7}=8.6 Hz)

7.30 (t, 2H, H-3′, H-5′, J_{H3′-H2′}=J_{H5′-H6}=7.8 Hz)

7.14 (d, 1H, H-9, J_{H9-H7}=1.9 Hz)

7.02 (t, 1H, H-4′, J_{H4′-H5′}=J_{H4′-H3′}=7.4 Hz)

6.83-6.88 (m, 3H, H-2′, H-6′, H-7)

4.48 (sl, 2H, H-a)

4.19-4.36 (m, 2H, H-1)

3.70 (dl, 1H, H-4-e, J_{H4e-H4a}=10.6 Hz)

3.27-3.42 (m, 2H, H-3e H-4-a)

3.09-3.01 (dl, 1H, H-3a, J_{H3a-H3e}=16.2 Hz)

¹³C-NMR (DMSO d6): δ (ppm)

158.7 (1C, C-1′)

148.4 (1C, C-8)

132.9 (1C, C-5a)

131.7 (1C, C-4-a)

131.1 (2C, C-2″ C-6″)

129.7 (1C, C-1″)

129.4 (2C, C-3′, C-5′)

129.2 (1C, C-4″)

128.5 (2C, C-3″ C-5″)

125.1 (1C, C-9a)

121.6 (1C, C-4′)

116.4 (2C, C-6′, C-2′)

114.5 (1C, C-7)

112.0 (1C, C-6)

108.5 (1C, C-9)

108.0 (1C, C-9b)

57.6 (1C, C-a)

48.2 (1C, C-3)

47.5 (1C, C-1)

19.9 (1C, C-4)

IR (KBr): v (cm⁻¹)

3158 (NH), 2654 (CH₂), 2528 (NH⁺Cl⁻), 1586 (C═C), 1487 (CH₂), 1235 (C—O—C or C—C—N)

Microanalysis: C₂₄H₂₂N₂OCl

Theory % C, 73.74; % H, 5.93; % N, 7.17; % O, 4.09; % Cl 9.07

Measured % C, 73.29; % H, 5.95; % N, 7.35; % O, 4.46; % Cl, 9.37

### Results of the Pharmacological Studies:

The results of the pharmacological studies are given below.
1. The inhibitory power of the molecules on inflammation induced by the stimulation of the saphenous nerve has been measured at follows

24 hours after a treatment with guanethidine (20 mg/kg, sc), the male Wistar rats (220-250 g) are anaesthetized with sodic pentobarbital (60 mg/kg, ip). The two back legs are shaved. After a cut in the upper part of the thigh, the saphenous nerve is cleared, cut, placed on a platinum electrode and immersed in a drop of paraffin oil. Only the electrode placed on the right leg is connected to a stimulator. This latter represents the “stimulated” leg by contrast to the “sham” left leg. The product (at a dosage of 5 μg/kg) or the corresponding solvent (NaCl 9 ‰-DMSO) are administrated via the jugular vein, 15 minutes before the electrical stimulation (ES). A plasmatic marker, Evans Blue (20 mg/kg iv) is administered through the penis vein 5 minutes before the ES. The saphenous nerve is stimulated according to the following conditions: 3V; 5 Hz; 1 ms; 5 minutes (Harvard stimulator). At the end of the ES, a blood sample is realized by cardiac puncture and the skin of the edema of each leg (visualized by the extravasation of the Evans Blue) is sampled and weighted later. The animals are killed by anaesthetical overdose. The blood samples are centrifuged (3,000 rotations/minute, during 15 minutes). The plasma is then diluted to 1/100, in distilled water. The plasmatic marker is extracted from the skin biopsies according to the method of Beach and Steinetz (J. Pharmacol. Exp. Therap., 1961, 131, 400-406). The skins sampled on the back legs are placed in tubes with a ground neck containing 3 ml of hydrochoric acid (36%). They are then digested by a 2 hours hydrolysis, at 37° C. 3 ml of benzalkonium chloride (12.8%) are then added. After shaking and 30 minutes of rest, the colored marker is extracted by 7 ml of dichloromethane. The tubes are slowly and regularly shaken, during 1 hour. The aqueous phase (upper) is eliminated by sucking up, by means of a vacuum pump, and the organic phase is filtered on paper. The Evans Blue is measured in the plasma and after extraction of the skin biopsies by a spectrophotometrical method, at 620 nm. The plasmatic extravasation developed on each leg is expressed in μl of plasma/g of skin. The neurogenous edema induced by the stimulation of the saphenous nerve is given as being the difference of plasma volume between the “stimulated” leg and the “sham” leg. The results are obtained on the same day with a group of treated rats and a group of control rats. The inhibitory power of the tested compound is measured by the ratio average volume of edema of treated rats/average volume of edema of control rats and expressed as percent.
2. The vascular contractile response has been measured with saphenous vein rings of rabbits (new Zealand, 2.5 to 3 kg) bound to a sensor, installed in organs baths (EMKA Technologies), containing a physiological Krebs-Henseileit solution. The reactivity of the isolated organ is controlled by means of an observation of one contraction induced by means of 100 mmol/l KCl followed by the observation of one relaxation induced by means of increasing concentrations of acetylcholine (0.1 to 10 μmol/l). The vasoconstrictive effect of the molecules with increasing concentrations is evaluated with saphenous vein rings contracted in an intermediate manner with 40 mmol/l KCl and treated with 0.5 μmol/l pargyline, an inhibitor of monoamine oxydase. The results are expressed as percent of the maximum effect induced by serotonine at 1 μmol/l. Under these conditions, the responses induced by agonists of 5HT1B/5HT1D, such as sumatriptan and/or 5-carboxamidotriptan are at a maximum.
3. The binding on the 5HT1B/5HT1D receptors of isolated membranes of bovine nuclear caudal has been measured by measuring the shifting of 5 nmoml/l of [³H]5-carboxamidotryptamine by increasing concentrations of the molecule in the presence of 100 nmol/l of 8-OH-dipropylaminotetralin (8-OH-DPAT) in order to mask the 5HT1A receptors and in the presence of 100 nM of mesulergine in order to mask the 5HT2 receptors. The concentration shifting 50% of the total binding of [³H]5-carboxamidotryptamine (IC₅₀ expressed in mol/l) is used here as the reactivity value.
4. The binding to 5HT1A receptors of isolated membranes of rat brain has been measured by measuring the shifting of 5 nmol/l of [³H] 8-OH-DPAT by increasing concentrations of the molecule in the presence of 100 nmol/l of mesulergine in order to mask the 5HT2 receptors. The concentration shifting 50% of the total binding of [³H] 8-OH-DPAT (IC50 expressed in mol/l) is used here as the reactivity value.

The results are given in the following table:

| | | Contraction. | | |
|---|---|---|---|---|
| | Neuro. Inf. | Saph. Vein | | IC₅₀ 5-HT1B/ |
| Example | Inhib. (%) | 10⁻⁶ mol/l | IC₅₀ 5-HT1A | 5-HT1D |
| N^{o} | 5 μg/kg iv | (%) | (mol/l) | (mol/l) |
| 1 | 65 | 0 | 3.10⁻⁵ | 1.10⁻⁵ |
| 2 | 60 | 0 | >10⁻⁵ | >10⁻⁵ |
| 3 | 80 | 0 | >5.10⁻⁶ | >5.10⁻⁶ |

The compounds of the present invention, injected intravenously at 5 μg/kg inhibit the plasmatic extravasation provoked by electrical stimulation of the saphenous nerve of the back legs of rats (neurogenic inflammation), without having a contractive effect upon the saphenous vein of rabbits at 10⁻⁶ mol/l and without binding, at physiological levels, with the receptors 5HT1B/5HT1D of bovine brain and 5HT1A of rat brain.

It has been found that the compounds of the present invention inhibit neurogenic inflammation, against the expectations of the skilled person, independent from a fixation to the receptors 5HT1A, 5HT1B and 5HT1D′. The absence of fixation to these receptors limits the risks secondary effects associated with those receptor subtypes, in particular vasoconstrictive effects (agonist effect 5HT1B/5HT1D) as observed with the triptans.

1. A compound of the Formula (I): 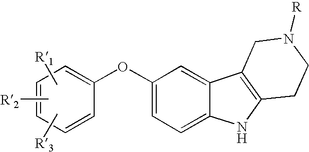
wherein
R represents hydrogen, a C₁-C₆ alkyl group, a C₁-C₆ haloalkyl group, a C₆-C₁₈ aryl group, a C₆-C₁₈ aryl-C₁-C₆ alkyl group, a heterocyclic group, a heterocycle-C₁-C₆ alkyl group, or a group wherein R forms with any one of the two carbon atoms adjacent to the nitrogen atom to which R is bound a condensed cyclic group, and wherein the alkyl group, the aryl group, the aralkyl group, the heterocyclic group and the condensed cyclic group may be substituted by one or more groups, selected independently from halogens, hydroxy, amino, monoalkylamino, dialkylamino, amido, N-alkyl amido, N,N-dialkyl amido, nitro, cyano, —COOH, —COO(C₁-C₄ alkyl), —OCF₃, —SO₂(C₁-C₄ alkyl), C₁-C₆ alkyl, C₁-C₆ alkoxy, phenoxy, C₁-C₆ alkoxy carbonyl, C₁-C₆ acyloxy, C₁-C₆ hydroxyalkyl, C₁-C₆ alkoxy-C₁-C₆ alkyl, C₁-C₆ alkyl carbonyl, and a residue comprising a linear chain with from 3 to 13 atoms selected from C and O, terminated with an aryl group,
R′₁, R′₂ and R′₃ are independently selected from hydrogen, halogens, hydroxy, nitro, cyano, —COOH, —COO(C₁-C₄ alkyl), —OCF₃, —SO₂(C₁-C₄ alkyl), C₁-C₆ alkyl, C₁-C₆ alkoxy, phenoxy, C₁-C₆ alkoxy carbonyl, C₁-C₆ acyloxy, C₁-C₆ hydroxyalkyl, C₁-C₆ alkoxy-C₁-C₆ alkyl and C₁-C₆ alkyl carbonyl, and pharmaceutically acceptable salts thereof.

2. The compound according to claim 1, wherein R is benzyl, optionally substituted by one or more substituents selected independently from the group consisting of halogen, nitro, cyano, —COOH, —COO(C₁-C₄ alkyl), —OCF₃, —SO₂ (C₁-C₄ alkyl), C₁-C₆ alkyl, C₁-C₆ alkoxy, C₁-C₆ alkoxy carbonyl, C₁-C₆ acyloxy, C₁-C₆ hydroxyalkyl, C₁-C₆ alkoxy-C₁-C₆ alkyl, C₁-C₆ alkyl carbonyl, and residue comprising a linear chain with from 3 to 13 atoms selected from C and O, terminated with an aryl group.

3. A compound of the formula

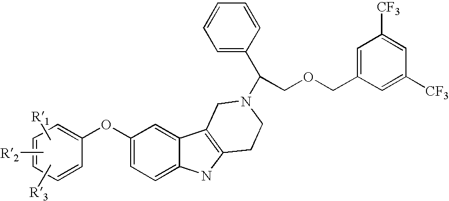

4. The compound according to claim 2, wherein R′₁, R′₂ and R′₃ represent hydrogen.

5. The compound according to claim 1 which is selected from the group consisting of phenyl-2,3,4,5,-tetrahydro-1H-pyrido[4,3-b]indole-8-yl ether (8-phenoxy-2,3,4,5-tetrahydro-1H-pyrido[4,3-b]indole, N-benzyl-8-phenoxy-2,3,4,5-tetrahydro-1H-pyrido[4,3-b]indol-8-yl ether (8-phenoxy-2-N-benzyl-2,3,4,5-tetrahydro-1H-pyrido[4,3-b]indole) and 2-benzyl-8-phenoxy-2,3,4,5-tetrahydro-5H-pyridinium[4,3-b]indole-hydrochloride.

6. (canceled)

7. A pharmaceutical composition, comprising at least one compound according claim 1 together with a pharmaceutically acceptable vehicle.

8. A method for the treatment and/or prevention of neurogenic inflammation comprising administering at least one compound according to claim 1 to an individual in need thereof in a therapeutic amount.

9. The method according to claim 8, wherein the inflammation to be treated is selected from the group consisting of:
Inflammation of the respiratory system (asthma, rhinitis, bronchiolitis, chemical and physical pollution, spondylitis rhematic, sarcose pulmonaire),
Hemorrhoidal inflammation,
Pelvic and urologic disorders (cystitis, incontinence),
pain (postraumatic, postchirurgic),
vascular inflammation/disorder (migraine, venous insufficiency),
digestive inflammation/disorder (vomiting, irritated colon),
inflammation of joints (arthritis, gout)
skin inflammation/irritation (psoriasis, urtikaria, dermatitis).
Cough

10. A pharmaceutical composition, comprising the compound according to claim 3 together with a pharmaceutically acceptable vehicle.

11. A method for the treatment and/or prevention of neurogenic inflammation comprising administering the compound according to claim 3 to an individual in need thereof in a therapeutic amount.

12. The method according to claim 11, wherein the inflammation to be treated is selected from the group consisting of:
Inflammation of the respiratory system (asthma, rhinitis, bronchiolitis, chemical and physical pollution, spondylitis rhematic, sarcose pulmonaire),
Hemorrhoidal inflammation,
Pelvic and urologic disorders (cystitis, incontinence),
pain (postraumatic, postchirurgic),
vascular inflammation/disorder (migraine, venous insufficiency),
digestive inflammation/disorder (vomiting, irritated colon),
inflammation of joints (arthritis, gout),
skin inflammation/irritation (psoriasis, urtikaria, dermatitis), and
Cough
